(19) United States
(12) Patent Application Publication
(10) Pub. No.: US 2024/0252571 A1
(43) Pub. Date: Aug. 1, 2024

(54) Hemp Essential Oil

(71) Applicant: Jiankang Luo, West Covina, CA (US)

(72) Inventor: Jiankang Luo, West Covina, CA (US)

(21) Appl. No.: 18/102,666

(22) Filed: Jan. 27, 2023

(51) Int. Cl. A61K 36/185; A61K 31/05; A61K 31/352; A61K 47/44

(52) U.S. Cl. CPC A61K 36/185; A61K 31/05; A61K 31/352; A61K 47/44

(57) ABSTRACT

Presented herein are methods and compositions for an improved natural essential oil skincare formulation. In a preferred embodiment of the present invention, the formulation comprises at least CBG, CBD, CBN, hemp oil, hemp seed oil, and coconut oil. The formulation may consist of other natural components at varying concentrations, depending on the purpose of its use. Also disclosed are methods of using the disclosed formulation. [symbol] referred method of use comprises identifying a condition to be treated and applying the composition disclosed herein to the skin to treat the condition. The methods and compositions disclosed herein may be used on humans or animals, for various topical uses.

### FIELD OF THE INVENTION

This invention relates generally to an essential oil formulation, more specifically, the present invention is an improved natural essential oil for topical treatment of various human conditions.

### BACKGROUND

Essential [symbol] are frequently used by individuals who seek to benefit from the safe and effective known results that can be obtained from using these natural ingredients. Many essential oils, however, are too harsh to be used topically and can cause irritation if applied directly on to the skin. Often, essential oils must be mixed or diluted to prevent unwanted side effects, thereby reducing their therapeutic effects.

Presently, most essential oil blends contain chemical compositions that can also be harsh on the skin or harmful to the health of the user, which counteracts the benefit gained from their use. Daily use of harmful chemicals over a prolonged period may result in serious health risks to consumers. Common artificial chemical compositions that are present in many essential oil blends include toxic parabens, carcinogenic nitrosamines, phthalates, imidazolidinyl urea, diazolidinyl urea, petroleum jelly, propylene glycol, PVP/VA copolymers, sodium lauryl sulfate, stearalkonium chloride, diethanolamine, artificial fragrances, artificial colors, allergens, and other irritants, to name a few. These chemical compositions can cause both immediate and long-term harm to users.

The immediate harm that may come from using artificial components include allergic reactions, contact dermatitis, headache, dizziness, eye irritation, hyper-pigmentation, allergic reactions, rosacea, hair loss, violent coughing, vomiting, and rashes. While the immediate harmful effects can be very disturbing, the long-term consequences of using these chemicals are much more alarming.

The long term harm that may be caused by using these chemicals may include systemic problems, such as endocrine disruptors, immune system toxicity, general toxicity, or cancer-causing agents. Microplastics found in some essential oil blends, particularly those containing scrubbing beads, can become lodged in lung tissue, leading to respiratory infections or disease. Some of these ingredients may also leech nutrients from the skin, promoting dry or damaged skin and become more vulnerable to sun damage.

In addition to the harm these chemicals may pose directly to the consumers, use of these toxic chemical compounds may also cause harmful effects to others, as well as to the environment. Chemicals and micro-plastics that are washed down the drain may end up in the general water supply, where they can affect others who have never used these products, as well as to animals, plants, and ecological systems as a whole.

While natural essential oil blends exist on the market, these products tend to be significantly less effective than those containing artificial ingredients. Most individual e [symbol] ntial oils are ineffective at calming irritation, redness, or eczema. While some essential oils may provide some anti-bacterial or antioxidant properties, very few are effective in treating a wide range of conditions. Consumers expect their products to function as required and will not tolerate products that are ineffective for their intended use.

What is needed is an effective, natural essential oil blend that is free of harmful chemicals to help the above situations.

The present invention solves this problem.

### SUMMARY OF THE INVENTION

Presented herein are formulations and methods for treating human conditions using an effective, all-natural essential oil composition. In one embodiment of the present invention, the composition comprises at least cannabigerol (CBG), cannabidiol (CBD), cannabinol (CNB), hemp oil, hemp seed oil, and coconut oil. In a preferred embodiment of the present invention, the composition further comprises an emulsification of at least three of the following components: cannabichromene (CBC), cannabidiolic acid (CBDA), cannabigerolic acid (CBGA), glycerin, Vaseline, lavender essential oil, chamomile essential oil, lemon essential oil, sweet orange essential oil, tea tree essential oil, basil essential oil, grape fruit essential oil, Litsea cubeba essential oil, citronella essential oil, cinnamon essential oil and peppermint essential oil.

While the benefits of each of these ingredients are well documented, the use of these components together as disclosed herein have produced an essential oil blend that is a significant improvement over the prior art. The present invention can be used to treat skin conditions, reduce anxiety, relieve pain, as a decongestant, as a mood enhancer, as a stress reducer, to calm, to induce sleep, as immunity support and to boost energy.

While some benefits of these individual essential oils are well known, it is the effect of carefully selected blends that was not well understood. As an emulsion, the blend of these essential oils creates a synergistic effect that produces unexpected results.

For example, it is well known that CBG can penetrate the basal and subcutaneous layers of the skin to calm inflammation, reduce blemishes, clean pores, balance sebum production and aid with cellular turnover.

CBD, as a topical agent, improves the overall health of the skin by improving nutrition, moisturizing, sterilization, anti-oxidation and decontamination of the treated skin. Studies have shown that CBD reduces inflammation, calms irritation and redness, and contains anti-bacterial properties. CBD also contains antioxidant properties, which protects the skin from damage, prevents inflammation and acne, soothes psoriasis and eczema, and contains anti-aging properties. CBD has also been used as a treatment for dermatitis.

Benefits of hemp oil include the decrease inflammation, provides relief for discomfort related to many skin conditions, including acne, psoriasis, eczema, and atopic dermatitis. Hemp oil also promotes the growth of new, healthy skin cells. It is suitable for most skin types and moisturizes without clogging pores. Balances oily skin, hydrates, regulates oil production. Softens skin. It is also a humectant, which draws environmental moisture to the skin and forms a protective barrier, which prevents moisture loss. Some studies show that hemp oil also firms and tightens skin.

Benefits of hemp seed oil include natural hydration for all skin types, reduction of inflammation and regulation of oil production. Hemp seed oil is also an excellent component to be used when preparing an essential oil blend since it does not interfere with the therapeutic properties of other essential oils.

Benefits of CBN include sleep aid and pain management. CBN has shown sedative properties that could be used to treat insomnia. Pain relief, specifically for muscle and joint pain is a another significant benefit of CBN. As a topical agent, CBN is also known to significantly improve the appearance of skin injuries, such as blisters. CBN is used as a treatment for epidermolysis, which are conditions that cause the skin to blister easily. CBN is well tolerated in both normal, intact skin, as well as on open wounds.

Benefits of coconut oil include anti-bacterial, anti-viral and anti-parasitic properties. These beneficial properties are derived from the richness of medium-chain fatty acids, which include lauric acid, octanoic acid, decanoic acid, and myristic acid. These fatty acids convert into antibacterial substances in the body, which resist harmful, invading microorganisms. Coconut oil has also demonstrated other topical and systemic benefits, including various therapeutic uses. Coconut oil may be also used to prevent injury, including bed rashes or other conditions caused by prolonged periods in one position.

The present invention can be used to calm irritation, redness or eczema of the dermis. The present invention may also be used as an anti-bacterial agent, antioxidant, skin care, remedy, anti-anxiety, improve mood, improve sleep, reduce odor, athlete's foot, and insect repellant, especially for acarid and mosquitoes.

The numerous beneficial properties of compositions disclosed herein allow it to be used for other purposes aside from cleansing the skin. For example, long distance drivers may use the composition disclosed in the present invention to prevent rashes or irritation that may develop from being seated for hours in the same position. In another example, campers without access to bathing facilities may utilize the present invention to clean their bodies to prevent discomfort or unpleasant odors. Maintaining cleanliness not only affects a person's physical health but may also improve their mental health and thereby improve the effectiveness of their jobs.

Benefits of the present invention include treat [symbol] the skin while relieving skin sensitivity and redness. This composition also provides a protective layer for the skin's surface and improves the skin's ability to self-heal. and skin care remedy, anti-anxiety and improving mood, improving sleep, reducing odor, athlete's foot and insect repellent.

In addition to protection, the composition disclosed herein may also treat a variety of skin conditions, including eczema, rashes, or dryness.

[symbol] formulation may also be used to repel insects and other vectors from humans or pets, thereby preventing infection, inflammation, or unsanitary conditions, resulting in foul odors.

The raw materials used for this formulation are all edible, natural raw plant materials or extracts. No chemicals or synthetic components are added to the invention.

The present invention is particularly suited for skin care protection, including, but not limited to moisturizing; calming irritation, redness, and eczema; anti-bacterial properties; antioxidant properties; anti-anxiety and improving mood; improving sleep; reducing odor; treatment of athlete's foot; and as an insect repellent. The composition disclosed herein also emits a pleasant, natural fragrance, thereby eliminating the need for additional artificial or irritating components.

The present invention also discloses methods of manufacturing and using the composition disclosed herein. In a preferred embodiment, a method of manufacturing the composition disclosed herein comprises combining hemp oil, hemp seed oil and coconut oil, heating the mixture, and then at lease CBD, CBG, CBN, Glycerin and Vaseline are added after emulsification.

In another preferred embodiment of the present invention, a method of using the composition disclosed herein comprises applying the composition disclosed herein onto a person or animal's skin.

This summary is not intended to disclose every embodiment or every aspect of the present invention. Rather, it is intended to provide an exemplification of some the concepts and features set forth herein. The features and advantages of this disclosure will be readily apparent from the following description of drawings and detailed description of the invention. This disclosure expressly includes any and all combinations and sub-combinations of the elements and features presented herein.

### DETAILED DESCRIPTION OF THE INVENTION

In the following description, for purposes of explanation, numerous specific details are set forth in order to provide a thorough understanding of the invention. It will be apparent, however, to one skilled in the art, that the invention can be practiced without each specific detail.

Reference in the specification to “one embodiment” or “an embodiment” means that a particular feature, structure, or characteristic described in connection with the embodiment is included in at least one embodiment of the invention. The appearances of the phrase “in one embodiment” in various places in the specification are not necessarily all referring to the same embodiment.

As used herein, the words or terms used herein have the following definitions:
“Approximately” or “about” mean±10%.
“CBG” means cannabigerol.
“CBD” means cannabidiol.
“CBN” means cannabinoid.
“Hemp oil” means oil obtained from pressing the stalk of a hemp plant.
“Hemp seed oil” means oil obtained from pressing hemp seeds.
“Coconut oil” means oil obtained from pressing the fruit of a coconut palm.

The composition comprises at least CBG, CBD, CBN, hemp oil, hemp seed oil, and coconut oil. In a preferred embodiment of the present invention, the composition further comprises an emulsification of at least three of the following components: CBC, CBDA, CBGA, Glycerin, Vaseline, Lavender essential oil, Chamomile essential oil, Lemon essential oil, Sweet Orange essential oil, Tea Tree essential oil, Basil essential oil, Grape Fruit essential oil, Litsea cubeba essential oil, Citronella essential oil, Cinnamon essential oil and Peppermint essential oil.

In a preferred embodiment, the present invention comprises CBG is present in an amount between 0.1% and 5%, CBN is present in an amount between 0.1% and 5%, CBD is present in an amount between 2.5% and 20%, hemp oil is present in an amount between 5% and 50%, hemp seed oil is present in an amount between 10% and 40%, and coconut oil is present in an amount between 10% and 40%.

In yet another preferred embodiment, the present invention comprises CBG in an amount between 0.1% and 5%, CBN is present in an amount between 0.1% and 5%, CBD is present in an amount between 2.5% and 20%, hemp oil is present in an amount between 5% and 50%, hemp seed oil is present in an amount between 10% and 40%, coconut oil is present in an amount between 10% and 40%, and at least three other components are present in an emulsification that comprises between 5% and 20% of the final composition. The emulsification comprises at least three of the following ingredients: CBC, CBDA, CBGA, Glycerin, Vaseline, lavender essential oil, chamomile essential oil, lemon essential oil, sweet orange essential oil, tea tree essential oil, basil essential oil, grape fruit essential oil, Litsea cubeba essential oil, citronella essential oil, cinnamon essential oil and peppermint essential oil.

In yet another preferred embodiment, the present invention comprises CBG in an amount between 0.1% and 5%, CBN is present in an amount between 0.1% and 5%, CBD is present in an amount between 2.5% and 20%, hemp oil is present in an amount between 5% and 50%, hemp seed oil is present in an amount between 10% and 40%, coconut oil is present in an amount between 10% and 40%, and at least two other components are present in an emulsification that comprises between 5% and 20% of the final composition. The emulsification may further comprise at least two of the following ingredients: CBC, CBDA, CBGA, glycerin, Vaseline, lavender essential oil, chamomile essential oil, lemon essential oil, sweet orange essential oil, tea Tree essential oil, basil essential oil, grape fruit essential oil, Litsea cubeba essential oil, Citronella essential oil, Cinnamon essential oil and Peppermint essential oil.

In yet another preferred embodiment, the present invention comprises CBG in an amount between 0.1% and 5%, CBN is present in an amount between 0.1% and 5%, CBD is present in an amount between 2.5% and 20%, hemp oil is present in an amount between 5% and 50%, hemp seed oil is present in an amount between 10% and 40%, coconut oil is present in an amount between 10% and 40%, and at least one other component is present in an emulsification that comprises between 5% and 20% of the final composition. The emulsification comprises at least one of the following ingredients: CBC, CBDA, CBGA, glycerin, Vaseline, lavender essential oil, chamomile essential oil, lemon essential oil, sweet orange essential oil, tea tree essential oil, basil essential oil, grape fruit essential oil, Litsea cubeba essential oil, citronella essential oil, cinnamon essential oil and peppermint essential oil.

In a preferred embodiment for pet use, the present invention comprises CBG is present in an amount between 0.1% and 5%, CBN is present in an amount between 0.1% and 5%, CBD is present in an amount between 2.5% and 20%, hemp oil is present in an amount between 5% and 50%, hemp seed oil is present in an amount between 10% and 40%, and coconut oil is present in an amount between 10% and 40%. Other ingredients may constitute 5% and 20%

Several studies have been conducted to determine the benefits of components of the present invention. As disclosed in Oláh A, Tóth BI, Borbíró I, et al. Cannabidiol exerts sebostatic and anti-inflammatory effects on human sebocytes. J Clin Invest. 2014; 124(9):3713-3724. doi:10.1172/JCI64628, investigation of the cutaneous cannabinoid system seems to be a promising choice when searching for novel therapeutic possibilities. Indeed, we have shown previously that the skin ECS regulates cutaneous cell growth and differentiation, and it reportedly exerts anti-inflammatory effects. Of further importance, we have also demonstrated that the ECS plays a key role in the regulation of sebum production. According to our recent findings, prototypic eCBs, such as N-arachidonoyl ethanolamide (anandamide [AEA]) and 2-arachidonoylglycerol, are constitutively produced in human sebaceous glands. Moreover, using human immortalized SZ95 sebocytes, we have also demonstrated that these locally produced eCBs (acting through a CB2 cannabinoid receptor→ERK1/2 MAPK→PPAR pathway) induce terminal differentiation of these cells, which is characterized by increased neutral lipid (sebum) production of the sebocytes. These findings confirmed unambiguously that human sebocytes have a functionally active ECS; yet we did not possess data on the potential effect(s) of plant-derived cannabinoids.

(−)-Cannabidiol (CBD) is the most studied nonpsychotropic phytocannabinoid. It has already been applied in clinical practice without any significant side effects (Sativex), and numerous ongoing phase II and III trials intend to explore its further therapeutic potential. Hence, within the confines of the current study, we intended to reveal the biological actions of CBD on the human sebaceous gland. Since we lack adequate animal models, we used human immortalized SZ95 sebocytes, the best available cellular system, and the full-thickness human skin organ culture (hSOC) technique.

CBD normalizes “pro-acne agent”-induced excessive lipid synthesis of human sebocytes. We first assessed the biological effects of CBD (1-10 μM) on the lipogenesis of SZ95 sebocytes. Although eCBs are known to show intense lipogenic actions via the metabotropic CB2 receptors, neither semiquantitative Oil Red O nor quantitative Nile Red staining indicated changes in the basal neutral (sebaceous) lipid synthesis of SZ95 sebocytes following 24-hour CBD treatment. Intriguingly, however, CBD markedly inhibited the lipogenic action of the prototypic eCB, AEA, in a dose-dependent manner.

We also tested its effect on actions of other lipogenic substances, which were shown previously to act through different, ECS-independent signal transduction mechanisms. Indeed, CBD effectively inhibited lipid synthesis induced by either arachidonic acid (AA) or the combination of linoleic acid and testosterone (LA-T), indicating that the effect of CBD is not “ECS specific” but a “universal” lipostatic action.

Since cannabinoids have been very often shown to exert “biphasic” effects (i.e., opposing physiological actions at nM vs. M concentrations), we also tested the effects of lower (1-100 nM) CBD concentrations; notably, they did not influence either basal or AA-induced lipid synthesis of the sebocytes.

We also investigated the effects of CBD on the lipidome of SZ95 sebocytes under in vitro conditions that mimicked “acne-like” circumstances (the latter was achieved by using a key “pro-acne” inflammatory mediator, AA). Importantly, CBD almost completely normalized the AA-enhanced “pathological” lipogenesis of SZ95 sebocytes. This suggests that CBD may primarily normalize both quantitatively and qualitatively excessive and abnormal lipid production induced by acne-promoting stimuli.

CBD decreases proliferation, but not the viability, of human sebocytes both in vitro and ex vivo. Besides the above lipostatic action, another desired effect of a proper anti-acne agent would be to inhibit the unwanted growth of sebocytes. Of great importance, proliferation of SZ95 sebocytes was significantly reduced in the presence of CBD (1-10 μM). It should be noted, however, that CBD did not suppress the cell count below the “starting” number (measured at day 1), arguing for a “pure” antiproliferative effect. Indeed, the lack of its effects on the count of viable cells was further verified by showing that these concentrations of CBD did not decrease cellular viability or induce either apoptosis or necrosis of SZ95 sebocytes. Notably, administration of 50 μM CBD evoked apoptosis-driven cytotoxicity and, hence, led to decreased lipogenesis. Likewise, elongated application of 10 μM CBD (6-day treatments) also decreased cell number and lipogenesis.

Clinically, the key question is whether the above in vitro observations could be translated into significant sebostatic (i.e., lipostatic and antiproliferative) effects of CBD on human sebaceous glands in situ. To explore this on the preclinical level, the full thickness hSOC technique was used. These hSOC assays, which mimic the human sebaceous gland function in vivo as closely as this is currently possible on the ex vivo level, clearly demonstrated that application of CBD completely prevented the lipogenic action of AEA in situ and, in line with our long-term in vitro observations, decreased basal lipogenesis as well. Likewise, CBD markedly suppressed the expression of the proliferation marker MKI67. This suggests that CBD may also operate as a potent sebostatic agent in vivo when tested in appropriate clinical trials.

CBD exerts universal anti-inflammatory actions. We additionally found that CBD also prevented the “pro-acne” LA-T combination from elevating the expression of TNFA, a key cytokine in the pathogenesis of acne vulgaris. These data suggested that CBD may exert anti-inflammatory actions on human sebocytes (as had already been demonstrated for CBD in several other experimental models, such as diabetes, rheumatoid arthritis, etc.). Therefore, in order to confirm the putative universal anti-inflammatory action of the CBD on human sebocytes, we next assessed its effects by modeling both Gram-negative infections (applying the TLR4 activator LPS) and Gram-positive infections (using the TLR2 activator lipoteichoic acid [LTA]). We found that CBD completely prevented the above treatments from elevating TNFA expression. Moreover, CBD also normalized LPS-induced IL1B and IL6 expression (expression of these 2 cytokines was found not to be modulated by 24-hour LA-T or LTA treatment: data not shown). Taken together, these results strongly suggest that CBD's universal sebostatic action is accompanied by substantial anti-inflammatory effects, which would be very much desired in the clinical treatment of acne vulgaris.

Sebostatic (i.e., lipostatic and antiproliferative), but not anti-inflammatory, actions of CBD are mediated by the activation of transient receptor potential vanilloid-4 ion channels. Next, we dissected the molecular mechanism(s) that underlie the remarkable lipostatic effects of CBD. As expected, neither CB1- nor CB2-specific antagonists (AM251 and AM630) were able to antagonize the lipid synthesis-inhibitory action of CBD; hence, alternative options had to be considered.

First, we studied the effects of CBD on the ionic currents of SZ95 sebocytes. Using whole-cell patch-clamp configurations, membrane currents were elicited by voltage ramp protocols and then normalized to cell membrane capacitance at two different potentials, i.e., at −90 and +90 mV. CBD (10 μM) induced a mostly outwardly rectifying current and a positive shift in the reversal potential, arguing for the activation of certain cation channels upon CBD application.

It is well known that various cannabinoids can modulate the activity of certain transient receptor potential (TRP) channels, collectively referred to as “ionotropic cannabinoid receptors”. Moreover, we have shown recently that activation of TRP vanilloid-1 (TRPV1) on SZ95 sebocytes by capsaicin also exerts potent lipostatic actions. Therefore, we first systematically explored these candidate “CBD targets.”

We found that SZ95 sebocytes express TRPV1, TRPV2, and TRPV4 both at the mRNA and protein levels (Supplemental FIG. 4, A-C). Among these TRP channels, TRPV4 showed the highest mRNA levels by far (expression of TRPA1 and TRPM8 was below the detection limit; data not shown).

Since the 3 identified TRPs are nonselective cation channels that are most permeable to Ca²⁺, we studied the effects of CBD on the calcium homeostasis of the sebocytes. Using a fluorescent Ca²⁺-imaging technique, we found that CBD significantly increased the intracellular calcium concentration ([Ca²⁺ ]_{IC}) of SZ95 sebocytes. This action was equally antagonized by (a) the decrease of the extracellular Ca²⁺ concentration ([Ca²⁺ ]_{EC}); (b) the nonspecific TRP channel blocker ruthenium red; and, of great importance, (c) the TRPV4-specific antagonist HC067047 (HC). We have also shown that the suppression of [Ca²⁺]^{EC} or the coapplication of HC also prevented the lipostatic action of CBD; notably, the TRPV4 antagonist alone did not affect basal lipid synthesis

See Oláh A, Tóth BI, Borbíró I, et al. Cannabidiol exerts sebostatic and anti-inflammatory effects on human sebocytes. J Clin Invest. 2014; 124(9):3713-3724. doi:10.1172/JCI64628

Jin S, Lee MY. The ameliorative effect of hemp seed hexane extracts on the Propionibacterium acnes-induced inflammation and lipogenesis in sebocytes. PLoS One. 2018; 13(8): e0202933. doi: 10.1371/journal.pone.0202933, investigated the anti-microbial, anti-infla.mmatory, and anti-lipogenic effects of hemp (Cannabis sativa L) seed hexane extracts, focusing on the Propionibacterium acnes-triggered inflammation and lipogenesis. Hemp seed hexane extracts (HSHE) showed anti-microbial activity against P. acnes. The expression of iNOS, COX-2, and the subsequent production of nitric oxide and prostaglandin increased after infection of P. acnes in HaCaT cells, however, upon treating with HSHE, their expressions were reduced. P. acnes-induced expressions of IL-1β and IL-8 were also reduced. HSHE exerted anti-inflammatory effects by regulating NF-κB and MAPKs signaling and blunting the translocation of p-NF-κB to the nucleus in P. acnes-stimulated HaCaT cells. Moreover, P. acnes-induced phosphorylation of ERK and JNK, and their downstream targets c-Fos and c-Jun, was also inhibited by HSHE. In addition, the transactivation of AP-1 induced by P. acnes infection was also downregulated by HSHE. Notably, HSHE regulated inflammation and lipid biosynthesis via regulating AMPK and AKT/FoxO1 signaling in KGF-1-induced inflammation and lipogenesis of sebocytes. In addition, HSHE inhibited 5-lipoxygenase level and P. acnes-induced MMP-9 activity and promoted collagen biosynthesis in vitro. Thus, HSHE could be utilized to treat acne vulgaris, through its anti-microbial, anti-inflammatory, anti-lipogenic, and collagen-promoting properties.

Baswan S M, Klosner A E, Glynn K, et al. Therapeutic potential of cannabidiol (CBD) for skin health and disorders. Clin Cosmet Investig Dermatol. 2020; 13:927-942. doi:10.2147/CCID.S286411, investigated the potcrnia of cannabidiol for skin health and dermatological conditons. Because the ECS plays an important regulatory function in the skin, it is plausible that treatment with topical cannabinoids could be efficacious for certain disorders or skin health in general. However, most of the clinical evidence to date has focused on the effects of CBD and other cannabinoids when consumed, inhaled, or injected. There is limited research investigating the therapeutic potential for topical applications. Yet, there is evidence to suggest applying cannabinoids, and specifically CBD, topically may be a viable route of administration for certain conditions. Although CBD has a reasonable molecular weight (314.46 Da), its high log P value (lipid/water partitioning) of ˜6.3, poses unique challenges to its transdermal delivery. However, this challenge may be overcome if appropriate carrier systems are used, as seen with CBD being absorbed transcutaneously in preclinical models. In 2003, Lodzki et al reported successful transdermal delivery of CD) in a murine model by using ethosomal carriers. Similarly, Hammel et al investigated the efficacy of topically applied CBD (1-10%) in a gel format, specifically for reduction of inflammation-associated symptoms in a monoarthritic rat model, and found that it was well-absorbed, as the plasma concentration showed a linear relationship with the dose applied. In vitro diffusion studies using human tissue have demonstrated CBD's permeation potential. However, at present no clinical trials investigating the topical absorptive capability in humans have been identified. Further work is warranted to better understand the appropriate doses and delivery methods for therapeutic CBD skin applications.

Skin serves as a protective barrier against environmental insults which can lead to the generation of reactive oxygen species (ROS). Oxidative stress induces cell damage and can result in chronic inflammation if left unchecked. It is also implicated in skin disorders and skin aging. Keratinocytes are the main cell type in the epidermis and are particularly sensitive to environmental stressors.

The harmful accumulation ofROS is countered in healthy skin by activation of numerous defense mechanisms. Many of these systems are controlled by the master regulator of cellular antioxidant defense system, NRF2 (nuclear factor erythroid 2-like 2) and PPAR-γ. The stress-induced enzyme Hemeoxygenasel (MIOX1) is one of the key NRF2 target genes and exhibits antioxidant and anti-inflammatory properties. In in vitro studies, CBD has demonstrated the ability to induce expression of HMOX1 and other NRF2-regulated genes. One study done in Normal Human Epidermal Keratinocytes (NHEK), reported that CBD induced the expression of several NRF2 target genes, with HMOX1 being the most upregulated by CBD. In the same study, increased levels of HMOX1 and expression of proliferation and wound repair keratins 16 and 17 were observed in mice epidermis after topical application of CBD. In another in vitro study using human keratinocytes, researchers showed that CBD was able to penetrate the cells and balance the oxidative stress response resulting from UVB irradiation and hydrogen peroxide. They also demonstrated that CBD had a protective effect against the peroxide-induced reduction of polyunsaturated fatty acids in the cell membrane, helping to protect membrane integrity. There is evidence to suggest CBD can activate PPAR-γ, as well. Treating 2D and 3D) fibroblast cells with CBD resulted in activation of PPAR-t with a corresponding decrease in levels of NF-kB. Since HMOX1 and PPAR-γ play strong cytoprotective roles with anti-inflammatory, antioxidant, and anti-apoptotic properties, treatments regulating their expression could be beneficial for skin conditions characterized by inflammation and keratin disorders, such as eczema or atopic dermatitis.

Atopic Dermatitis (AD) is a chronic inflammatory skin disorder associated with multifactorial causes like environmental triggers, damaged skin barrier function, microbiome imbalance, genetic predisposition, and an altered immune response. Phytocannabinoids have been shown to modulate inflammatory responses by regulating more than one underlying mechanism. Adelmidrol, a PEA derivative, has been shown to be effective in treating mild AD in a pediatric population. Though the efficacy of CBD is yet to be clinically validated, in a recent study by Petrosino et al, CBD was shown to exhibit anti-inflammatory properties in an experimental, allergic contact dermatitis model.

The influence of microbiome imbalance, especially due to colonization and biofilm formation of Staphylococcus aureus (S. aureus), has also emerged as an influencing factor which can contribute towards the severity of dermatitis. The preliminary data indicating the antimicrobial and antibiofilm activity of hemp come from the essential oil (steam distillate) fraction of hemp which is composed mainly of terpenoids such as myrcene, α-pinene, β-caryophyllene and other terpenes, but no significant levels of CBD. Zengin et al evaluated the antimicrobial and antibiofilm efficacy of hemp essential oil (EO) against a reference strain (S aureus American Type Culture Collection (ATCC) 29,213) and three clinical strains (S. aureus 101 TV, S. aureus 104, and S. aureus 105). The effective Minimum Inhibitory Concentration (MIC), Minimum Bactericidal Concentration (IBC), and the Minimum Biofilm Eradication Concentration (MBEC) hemp EO values against all S. aureus strain types were reported as 8, 16 and 24 mg/mL, respectively, which indicated the hemp EO may disrupt and eradicate a mature biofilm of S. aureus. Thus, the antimicrobial and antibiofilm activities of hemp EO against S. aureus suggest its therapeutic potential to prevent skin disorders like atopic dermatitis.

When it becomes chronic, itch or pruritis can severely affect one's quality of life. The pathogenesis of pruritis is well researched and is described comprehensively in various recent review articles. Though most of the ECS research indicates that the itch response is primarily modulated through CB1 receptors in the CNS, some reports argue the involvement of peripheral CB1 receptors could also be a potent contributor to itch. The available data thus far for the involvement of peripheral CB2 receptors are conflicting and more research is needed to conclusively determine its role in pruritis. It has also been shown that all ionotropic cannabinoid responsive receptors (e.g., TRPV1-4, TRPA1 and TRPM8) play a vital role in the complex cutaneous communication between keratinocytes, immune (Mast) cells and the sensory nerves which leads to an itch sensation. Thus, inhibiting the activity of such ionotropic channels by selective PCBs may be helpful in alleviating pruritis.

FAAH and MAGL inhibitors, which can increase the levels of endocannabinoids and modulate cannabinoid and non-cannabinoid receptor responses, were found to demonstrate anti-pruritic effects on murine models when administered via intraperitoneal and intrathecal routes. Though cannabinoids like THC and PEA have been shown to reduce itching in murine models, the human clinical data for testing the antipruritic potential of PEA have resulted in conflicting results. To add to the dilemma, a study by Spradley et al indicated that peripheral endocannabinoids have opposite effects on itching behavior in spinally versus trigeminally innervated skin of mice, and therapeutic treatment of itch might be more relevant for treating the lower body than itch arising from trigeminal innervated skin of the face or scalp. Since CBD is a FAAH inhibitor, a CB2 inverse agonist (antagonist of CB2 agonists) and TRPV1 agonist, it could potentially play a role in modulating itch response, but the scientific evidence remains scarce for this application to-date.

The major factors involved in acne onset are sebum overproduction, unwanted sebocyte proliferation, and inflammation. It is known that the ECS plays a key role in homeostasis of the skin, and specifically in lipogenesis. The endocannabinoid AEA, has been shown to stimulate lipid production in human sebocytes at low concentrations but induces apoptosis at higher levels. Although current research is limited, several in-vitro studies indicate that CBD could be a novel therapeutic in the management of acne by acting on pathways relating to sebum production, sebocyte proliferation, and inflammation. One notable study performed by Oláh et al addresses CBD's potential effects on several of these outcomes. First, researchers investigated the effects of CBD on sebaceous gland function in human SZ95 cells. They found that a 24-hour treatment of CBD (1-10 μM) alone caused no changes in cellular lipid synthesis; however, when cells were first treated with ALA, CBD was able to quell the lipogenic actions in a dose-dependent manner. The researchers went on to test other lipogenic substances, including arachidonic acid and a mixture of linoleic acid and testosterone, and found that CDI) was able to inhibit extraneous lipid synthesis induced by those compounds, as well. This finding suggests that CBD's effect is a universal action and is not limited to direct ECS interaction. Also, it is important to note that CBD does not simply reduce lipid production but rather it is able to normalize lipogenesis in a state of imbalance. The same researchers went on to investigate the anti-proliferation abilities of CBD in-vitro. They found that CBD did not suppress cell counts beyond the starting number (did not reduce the number of viable cells) but did significantly reduce the overall proliferation of cells at 1-10 μM doses. Higher doses of CBD (50 μM) or elongated application (6 days) did result in apoptosis-driven cytotoxicity and overall viable cell count was reduced.

Finally, Oláh's research group examined CBDs anti-inflammatory actions and found that it was able to prevent pro-acne mediators from elevating TNF-α mnRNA expression. It also was able to normalize the LPS-induced expression of IL-1B and IL6. This data provided further evidence for CBD's substantial anti-inflammatory actions. Notably, it is believed the control of sebocyte proliferation and lipid production was mediated through TRPV4 signaling, while the anti-inflammatory effects of CBD application were not.

In addition to previously mentioned factors, imbalance in the skin microbiome may also contribute to the pathogenesis of acne. Specifically, Cutibacterium acnes (C. acnes) overgrowth has been linked to the establishment of acne for over 100 years. Therefore, the known anti-microbial effects of (31) may also prove effective in acne treatment. In an in-vitro study by Jin et al, a hemp seed hexane extract (HSHE) exhibited anti-microbial activity on C. acnes while inducing inflammation, and lipogenesis in sebocytes at the molecular and cellular level. With 20% HSHE treatment, complete inactivation of C. acnes was observed. In this study, the content of CBD in HSHE was not reported; hence, it is difficult to attribute the contribution of CBD alone towards inactivation of C. acnes. Similarly, in a small clinical study involving men with buccal facial acne, a 3% Cannabis seed extract containing cream led to decreased sebum content and erythema. As cannabis seed extract contains minimal CBD content, it limits our understanding of application of CBD for acne and seborrhea therapy. Likewise, hemp essential oil contains many terpenes which were shown to have anti-microbial effects against C. acnes (formerly known as Propionibacterium acncs).

We speculate that Hemp seed extract or hemp EO could also have potential for treating acne vulgaris because of its anti-lipogenic, anti-proliferative, anti-inflammatory, and anti-microbial, properties, which may target similar or independent mechanisms than that of CBD. Unfortunately, no large-scale human trials have investigated the role of CBD for the management of acne. Larger studies will help to understand how CBD may impact acne at the clinical level.

CBD and CBG have been reported to have potent activity against a variety of Gram-positive Methicillin-resistant Staphylococcus aureus (MRSA) strains. Likewise, an antimicrobial effect of CBD against Listeria monocytogenes, Enterococcus faecalis and Methicillin-resistant Staphylococcus epidermidis (MRSE) was reported with Minimum Inhibitory Concentration (MIC) values of 4 μg/mL for MRSA and L. monocytogenes and 8 μg/mL for E. faecalis and MRSE. This study also characterized CBD as a helper compound that potentiates the effect of Bacitracin (BAC), a skin antibiotic. The MIC value of BAC was remarkably reduced to at least a 64-fold reduction for MRSA, MRSE and E. faecalis, when combined with ½ MIC of C3D as compared to MIC of BAC alone.

Furthermore, to assess the potentiating and synergistic effect against MRSA, growth curve and time kill assay results showed the combined activity of CBD and BAC reduced bacterial viability by 6-log₁₀ cfu/mL as compared to CBD or BAC alone. Interestingly, CBD was able to potentiate the effects of BAC against MRSA (S. aureus USA300) and other Gram-positive bacteria. The spectrum of use of CBD and BAC on growth of Gram-negative bacteria, including Pseudomonas aeruginosa, Salmnonella typhimiurium, Klebsiella pnemoniae, and Escherichia coli, was also measured. The results obtained from the combined effect of CBD and BAC against these Gram-negative bacteria concluded that the combined activity of CBD and BAC was considered ineffective against Gram-negative bacteria. Due to potent antibacterial properties against Gram-positive bacteria, cannabinoids can be used as an effective helper compound when combined with known antimicrobial actives to fight antibiotic resistant Gram-positive bacteria which cause skin disorders and other infections

Atalay S, Jarocka-Karpowicz I, Skrzydlewska E. Antioxidative and anti-inflammatory properties of cannabidiol. Antioxidants (Basel). 2019; 9(1):21. doi:10.3390/antiox9010021

Cannabidiol (CBD) is one of the main pharmacologically active phytocannabinoids of Cannabis sativa L. CBD is non-psychoactive but exerts a number of beneficial pharmacological effects, including anti-inflammatory and antioxidant properties. The chemistry and pharmacology of CBD, as well as various molecular targets, including cannabinoid receptors and other components of the endocannabinoid system with which it interacts, have been extensively studied. In addition, preclinical and clinical studies have contributed to our understanding of the therapeutic potential of CBD for many diseases, including diseases associated with oxidative stress. Here, we review the main biological effects of CBD, and its synthetic derivatives, focusing on the cellular, antioxidant, and anti-inflammatory properties of CBD.

Perez E, Fernandez J R, Fitzgerald C, et al. In vitro and clinical evaluation of cannabigerol (CBG) produced via yeast biosynthesis: a cannabinoid with a broad range of anti-inflammatory and skin health-boosting properties. Molecules. 2022; 27:491. Doi:10.3390/molecules27020491

Cannabigerol (CBG) is a minor non-psychoactive cannabinoid present in Cannabis sativa L. (C. sativa) at low levels (<1% per dry weight) that serves as the direct precursor to both cannabidiol (CBD) and tetrahydrocannabinol (THC). Consequently, efforts to extract and purify CBG from C. sativa is both challenging and expensive. However, utilizing a novel yeast fermentation technology platform, minor cannabinoids such as CBG can be produced in a more sustainable, cost-effective, and timely process as compared to plant-based production. While CBD has been studied extensively, demonstrating several beneficial skin properties, there are a paucity of studies characterizing the activity of CBG in human skin. Therefore, our aim was to characterize and compare the in vitro activity profile of non-psychoactive CBG and CBD in skin and be the first group to test CBG clinically on human skin. Gene microarray analysis conducted using 31) human skin equivalents demonstrates that CBG regulates more genes than CBD, including several key skin targets. Human dermal fibroblasts (HDFs) and normal human epidermal keratinocytes (NHEKs) were exposed in culture to pro-inflammatory inducers to trigger cytokine production and oxidative stress. Results demonstrate that CBG and CBD reduce reactive oxygen species levels in HDFs better than vitamin C. Moreover, CBG inhibits pro-inflammatory cytokine (Interleukin-1β, -6, -8, tumor necrosis factor α) release from several inflammatory inducers, such as ultraviolet A (UVA), ultraviolet B (UVB), chemical, C. acnes, and in several instances does so more potently than CBD. A 20-subject vehicle-controlled clinical study was performed with 0.1% CBG serum and placebo applied topically for 2 weeks after sodium lauryl sulfate (SLS)-induced irritation. CBG serum showed statistically significant improvement above placebo for transepidermal water loss (TEWL) and reduction in the appearance of redness. Altogether, CBG's broad range of in vitro and clinical skin health-promoting activities demonstrates its strong potential as a safe, effective ingredient for topical use and suggests there are areas where it may be more effective than CBD.

Methods of using the composition disclosed herein are also claimed. For example, a preferred method of use comprises a method of cleansing skin, the method comprising applying the composition to the skin; and removing the composition from the skin.

It is understood that this invention is not limited to only the elements described herein and that other types of elements will be equivalent for the purposes of this invention. The invention has been described by referencing preferred embodiments and several alternative embodiments, however one of ordinary skill in the art understands that employing other variables and modifications does not depart from the spirit and the scope of the present invention.

Although the invention has been disclosed in terms of specific embodiments herein, in light of these teachings, one of ordinary skill in the art may generate additional embodiments and modifications without departing from the spirit or the scope of the claimed invention. It is understood that the examples and descriptions disclosed herein are merely to facilitate understanding of the invention and should not be construed to limit the scope thereof.

It is also contemplated that the present invention may also apply in other industries, including, but not limited to, the sale of other types of goods.

1. A natural essential oil comprising:
CBG,
CBN,
CBD,
Hemp oil,
Hemp seed oil, and
Coconut oil.

2. The natural essential oil disclosed in claim 1, further comprising at least one of the following components: CBC, CBDA, CBGA, glycerin, Vaseline, lavender essential oil, chamomile essential oil, lemon essential oil, sweet orange essential oil, tea tree essential oil, basil essential oil, grape fruit essential oil, Litsea cubeba essential oil, citronella essential oil, cinnamon essential oil and peppermint essential oil.

3. The natural essential oil disclosed in claim 1, further comprising at least two of the following components: CBC, CBDA, CBGA, glycerin, Vaseline, lavender essential oil, chamomile essential oil, lemon essential oil, sweet orange essential oil, tea tree essential oil, Basil essential oil, Grape Fruit essential oil, Litsea cubeba essential oil, citronella essential oil, cinnamon essential oil and Peppermint essential oil.

4. The natural essential oil disclosed in claim 1, further comprising at least three of the following components: CBC, CBDA, CBGA, glycerin, Vaseline, lavender essential oil, chamomile essential oil, lemon essential oil, sweet orange essential oil, Tea Tree essential oil, Basil essential oil, Grape Fruit essential oil, Litsea cubeba essential oil, Citronella essential oil, Cinnamon essential oil and Peppermint essential oil.

5. The natural essential oil disclosed in claim 1, wherein CBG is present in an amount between 0.1% and 5%, CBN is present in an amount between 0.1% and 5%, CBD is present in an amount between 2.5% and 20%, hemp oil is present in an amount between 5% and 50%, hemp seed oil is present in an amount between 10% and 40%, and coconut oil is present in an amount between 10% and 40%.

6. The natural essential oil disclosed in claim 1, wherein CBG is present in an amount between 0.1% and 5%, CBN is present in an amount between 0.1% and 5%, CBD is present in an amount between 2.5% and 20%, hemp oil is present in an amount between 10% and 50%, hemp seed oil is present in an amount between 10% and 40%, coconut oil is present in an amount between 10% and 40%, and at least three other components are present in an emulsification that comprises between 5% and 20% of the final composition.

7. A method of treating skin, the method comprising:
identifying an area of the skin to be treated; and
applying the composition disclosed in claim 1 to the identified area.

8. The method disclosed in claim 7, further comprising removing the composition from the skin using a material, the material composed of a natural, synthetic, or combination of natural and synthetic materials.

9. The method disclosed in claim 7, further comprising removing the composition from the skin by rinsing with a liquid, wherein the liquid is water.

10. A method of treating a human condition, the method comprising:
identifying a condition to be treated; and
applying the composition disclosed in claim 1 to a specified area.

11. The method disclosed in claim 10, wherein the human condition is a skin condition.

12. The method disclosed in claim 7, wherein the skin condition is an animal skin condition.

13. The method disclosed in claim 10, wherein the human condition is irritated skin.

14. The method disclosed in claim 10, wherein the human condition is skin redness.

15. The method disclosed in claim 10, wherein the condition is eczema.

16. The method disclosed in claim 10, wherein the condition is caused by a microorganism.

17. The method disclosed in claim 10, wherein the condition is caused by an insect.

18. The method disclosed in claim 10, wherein the condition is anxiety or insomnia.

19. The method disclosed in claim 10, wherein the condition is athlete's foot.

20. The method disclosed in claim 10, wherein the condition is unpleasant odor.
